(19) United States
(12) Patent Application Publication
(10) Pub. No.: US 2007/0197592 A1
(43) Pub. Date: Aug. 23, 2007

(54) Use of Paliperidone for the Treatment of Substance Abuse

(76) Inventors: Sandra Boom, Dordrecht (NL); Marielle-Henriette Eerdekens, Dilsen-Stokkem (BE); Randall L. Morrison, Erdenheim, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/534,636

(22) Filed: Sep. 22, 2006

Related U.S. Application Data
(60) Provisional application No. 60/749,680, filed on Dec. 12, 2005.

(51) Int. Cl. A61K 31/445; A61K 31/4178

(52) U.S. Cl. 514/317; 514/402

(57) ABSTRACT

The present invention relates to the use of paliperidone, its pharmaceutically acceptable acid addition salts, enantiomeric forms, or esters thereof, for the treatment of substance abuse in a patient in need thereof. The invention provides a pharmaceutical composition comprising a therapeutically effective amount of paliperidone, its pharmaceutically acceptable acid addition salts, enantiomeric forms, or esters thereof, together with a pharmaceutically acceptable carrier, for the treatment of substance abuse in a patient in need thereof. The invention also provides for the use of a pharmaceutical composition as defined above for the treatment of substance abuse in a patient, and for the use of this pharmaceutical composition in the manufacture of a medicament for the treatment of substance abuse in a patient in need thereof.

This application claims priority from and benefit of provisional patent application 60/749,680 filed Dec. 12, 2005.

### TECHNICAL FIELD

This invention relates to the use of paliperidone for the treatment of substance abuse. The present invention is in the general field of compositions and treatments for substance abuse, more particularly alcohol abuse.

### BACKGROUND

Alcohol abuse, typically characterized as a maladaptive pattern of alcohol use, leading to clinically significant impairment or distress, is a serious medical and social problem. It has been suggested that agents producing a selective decrease in alcohol drinking in animals, without producing a parallel decrease in water or food intake, are likely to be clinically effective in the treatment of human alcoholism (Myers 1994). Daidzin, the active ingredient of the Chinese herb Radix pureariea (RP), used as a traditional treatment for “alcohol addiction” in China, fits the profile: it decreases alcohol drinking in the golden hamster, without producing a decrease in water or food intake (Keung and Vallee 1993). In contrast, many drugs, including specific serotonergic agonist (e.g., sertraline) and opiate antagonists (e.g., naloxone and naltrexone), that have been shown to inhibit alcohol consumption in animals have also impaired water or food consumption at the same time (Myers 1994). However, although atypical antipsychotics have been proposed as possible treatments for substance abuse, there medication may undergo substantial hepatic metabolism in substance abuse patients. The population of patients with hepatic impairment is quite high. Consequently, it would be advantageous to treat substance abuse patients with an atypical antipsychotic, which was not significantly metabolized in the liver.

### SUMMARY

We have discovered that paliperidone (9-OH risperidone), an atypical antipsychotic medication, its pharmaceutically acceptable acid addition salts, enantiomeric forms and esters thereof alone or in combination with other medications is useful to treat alcohol or other substance abuse, particularly in the general (not suffering from another psychiatric disorder) population. Generally stated, one aspect of the invention features a method of treating a patient suffering from alcohol or other substance abuse by administering to the patient paliperidone, its pharmaceutically acceptable acid addition salts, enantiomeric forms or esters thereof effective to rectify an abuse-associated dysfunction in the DA-mediated brain reward circuit. The medication may be a single compound, paliperidone, its pharmaceutically acceptable acid addition salts, enantiomeric forms, or esters thereof, or it may include a second compound which together achieve the specified function. For example, the medication may include a first component paliperidone (its pharmaceutically acceptable acid addition salts, enantiomeric forms, or esters thereof) and a second component which is an α2 receptor antagonist. One example of a suitable second component is idazoxan.

In one embodiment, the invention provides for the use of a pharmaceutical composition comprising a therapeutically effective amount of paliperidone, its pharmaceutically acceptable acid addition salts, enantiomeric forms, or esters thereof, together with a pharmaceutically acceptable carrier, for the treatment of substance abuse in a patient in need thereof.

In another embodiment, the invention provides for the use of a pharmaceutical composition comprising a therapeutically effective amount of paliperidone, its pharmaceutically acceptable acid addition salts, enantiomeric forms, or esters thereof, together with a pharmaceutically acceptable carrier, in the manufacture of a medicament for the treatment of substance abuse in a patient in need thereof.

In still another embodiment, the invention provides a pharmaceutical composition comprising a therapeutically effective amount of paliperidone, its pharmaceutically acceptable acid addition salts, enantiomeric forms, or esters thereof, together with a pharmaceutically acceptable carrier, for the treatment of substance abuse in a patient in need thereof.

In another embodiment, the patient is not being simultaneously treated for a psychiatric condition other than substance abuse. In yet another embodiment, the treatment is for alcohol abuse.

The details of one or more embodiments of the invention are set forth in the description below. Other features, objects and advantages of the invention will be apparent from the description and from the claims.

### DETAILED DESCRIPTION

As noted, the invention generally features methods of treating substance abuse and alcohol abuse in particular. The medications used in the invention may include a first component paliperidone (its pharmaceutically acceptable acid addition salts, enantiomeric forms, or esters thereof) and a second component which is an α2 receptor antagonist. As mentioned above, one example of a suitable second component is idazoxan. The patients to be treated according to the invention are those with a history or a risk of alcohol abuse, according to DSM-IV.

Paliperidone, including its pharmaceutically acceptable acid addition salts, enantiomeric forms, and esters, may be administered for the practice of the present invention. Paliperidone is well known in the art and is described in U.S. Pat. No. 5,158,952, which is hereby incorporated by reference.

As noted in U.S. Pat. No. 5,158,952, paliperidone has basic properties and, consequently, this compound may be converted to its therapeutically active non-toxic acid addition salt forms by treatment with appropriate acids, such as, for example, inorganic acids, such as hydrohalic acid, e.g. hydrochloric, hydrobromic acid and the like, sulfuric acid, nitric acid, phosphoric acid and the like; or organic acids, such as, for example, acetic, propanoic, hydroxyacetic, 2-hydroxypropanoic, 2-oxopropanoic, ethanedioic, propanedioic, butanedioic, (Z)-2-butenedioic, (E)-2-butenedioic, 2-hydroxybutanedioic, 2,3-dihydroxybutanedioic, 2-hydroxy-1,2,3-propanetricarboxylic, methanesulfonic, ethanesulfonic, benzenesulfonic, 4-methylbenzenesulfonic, cyclohexanesulfamic, 2-hydroxybenzoic, 4-amino-2-hydroxybenzoic and the like acids. Conversely the salt form can be converted into the free base form by treatment with alkali. The term acid addition salt as used hereinabove also comprises the solvates which such compounds are able to form and said solvates are meant to be included within the scope of the present invention. Examples of such solvates are e.g., the hydrates, alcoholates and the like.

Esters of Paliperidone are known in the art and are described in U.S. Pat. No. 5,254,556. Esters of paliperidone include octanoic acid, decanoic acid, dodecanic acid, tetradecanoic acid or hexadecanoic acid (palmitic acid). The currently preferred ester of palipidone is paliperidone palmitate.

Alcoholic patients that are suspected of hepatic impairment can be identified by examination of their medical records, taking their histories, physical examination or by laboratory testing. Physicians and nurses treating psychiatric patients should be familiar with the symptoms and tests for impaired liver functions. For example patients presenting with symptoms such as jaundice, liver palms, cerebral oedema, etc. should be further examined for liver impairment. Laboratory tests showing thrombocytopenia, raised bilirubin, low pseudocholinesterase, elevated lactate, raised creatinine etc. which should be further investigated. Appropriate techniques to determine whether there is impairment of liver function are known in the art. Normally a battery of tests will be run such as test for the levels of transaminase (e.g. aspartate aminotransferase, alanine aminotransfrease, etc.) and γ-glutamyltransferase, Hepatitis C serologies, Hepatitis B serologies Hepatitis A serology, Ceruloplasmin, serum protein electrophoresis, hepatic sonogram prothrombin time, CBC with platelet count and serum albumin.

It is important to recognize that such a treatment might also be an important treatment for substance abuse in general, since most substances of abuse act on DA circuits in a manner quite similar to that of alcohol. Other such substances of abuse are: cannabis, amphetamines and cocaine.

The compounds to be administered can be formulated into a suitable pharmaceutical preparation by known techniques, for example well known tablets and capsule formulations. Such formulations typically comprise the active agent (or the agent in a salt form) and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include oral, intravenous, intradermal, subcutaneous, transdermal (topical), transmucosal (e.g intranasal), and rectal.

By far the most convenient route of administration is oral (ingestion). Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

It is especially advantageous to formulate oral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Paliperidone may be formulated with pharmaceutical excipients into a variety of dosage forms as described in U.S. Pat. No. 5,158,952. Paliperidone will in one embodiment of the present invention be provided in an oral dosage forms. Suitable oral dosage forms include but are not limited to tablets, pills, fast dissolving dosage forms, controlled release or extended release dosage forms. Currently preferred are extended release OROS oral dosage forms which are well known in the art. Examples of oral dosage forms of paliperidone are described in US 20040092534, US 20050208132 and US 20050232995, which are all hereby incorporated herein by reference.

Paliperidone palmitate, including its pharmaceutically acceptable acid addition salts, and stereoisomeric forms, is also well known in the art and may also be formulated with pharmaceutical excipients into a variety of dosage forms as described in U.S. Pat. No. 5,254,556. Currently, it is preferred to administer paliperidone palmitate in a depot.

Paliperidone palmitate is considered to be a potentially valuable prodrug of paliperidone. A pharmaceutical composition suitable as an injectable solution of paliperidone palmitate may comprise a formulation of paliperidone palmitate in an appropriate oil for prolonged action; for example, peanut oil, sesame oil, cottonseed oil, corn oil, soy bean oil, synthetic glycerol esters of long chain fatty acids and mixtures of these and other oils.

In another embodiment, a pharmaceutical composition suitable as an efficient, well-tolerated, sustained or delayed release (depot) formulation for administration of paliperidone palmitate by intramuscular or subcutaneous injection may comprise a suspension of paliperidone palmitate in aqueous solution. Ideally, suitable aqueous depot formulations will comprise as much prodrug as can be tolerated so as to keep the injected volume to a minimum, and as little of the other ingredients as possible. In particular, such a composition will comprise by weight based on the total volume of the composition:
(a) from 3 to 20% (w/v) of the prodrug;
(b) from 0.05 to 2% (w/v) of a wetting agent;
(c) one or more buffering agents;
(d) from 0.5 to 2% (w/v) of a suspending agent;
(e) up to 2% (w/v) preservatives; and
(f) water q.s. ad 100%.

In yet another embodiment, the above composition may comprise a dispersion of particles consisting essentially of a therapeutically effective amount of crystalline paliperidone palmitate having a surfactant absorbed to the surface thereof in an amount effective in maintaining a specific surface area>4 m2/g (corresponding to an effective average particle size of less than 2,000 nm), in a pharmaceutically acceptable carrier comprising water. Preferably, the specific surface area is>6 m2/g, and in particular is in the range from 10 to 16 m2/g. Useful surface modifiers are believed to include those which physically adhere to the surface of the paliperidone palmitate but do not chemically bond thereto. Suitable surface modifiers can preferably be selected from known organic and inorganic pharmaceutical excipients. Such excipients include various polymers, low molecular weight oligomers, natural products and surfactants. Preferred surface modifiers include nonionic and anionic surfactants. Most of these excipients are described in detail in the Handbook of Pharmaceutical Excipients, published jointly by the American Pharmaceutical Association and The Pharmaceutical Society of Great Britain, the Pharmaceutical Press, 1986. The surface modifiers are commercially available and/or can be prepared by techniques known in the art. Two or more surface modifiers can be used in combination.

Suitable aqueous depot formulations such as those described above are well known in the art and specific details are provided in U.S. Pat. No. 6,077,843, U.S. Pat. No. 6,320,048 and U.S. Pat. No. 6,555,544, which are all incorporated herein by reference. Typically, suitable aqueous depot formulations will be administered approximately every three weeks or even at longer intervals where possible. The dosage should range from about 2 to 4 mg/kg body weight.

The term “therapeutically effective amount” as used herein, means that amount of active compound or pharmaceutical agent that elicits the biological or medicinal response in human that is being sought by a researcher, medical doctor or other clinician, which includes alleviation of the symptoms of the disease or disorder being treated.

Those of skill in the treatment of substance abuse could easily determine the effective amount of paliperidone to administer. In general it is contemplated that an effective amount would be from about 0.01 mg/kg to about 2 mg/kg body weight. In one embodiment of present invention, paliperidone is orally administered in a dosage form to a subject once a day. The mg of compound delivered in such a dosage form to the patient may be from 0.25 to about 20 mg (e.g. 0.25 mg, 0.5 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, and 20 mg) per oral dosage form.

The pharmaceutical compositions can be included in a container, pack, or dispenser together with instructions for administration.

I. Prophetic Examples

Methods. Twenty adult male Syrian golden hamsters are given access to alcohol in a free choice condition for 24 days prior to drug treatment. Animals are treated with either paliperidone (2 mg/kg for 2 days and 4 mg/kg for 7 days) haloperidol (0.2 mg/kg for 2 days and 0.4 mg/kg for 7 days) or vehicle (s.c.) on a daily basis for 9 days and daily consumption of alcohol, water and food is recorded, as is body weight, by a technician blinded to treatment group. Following a 9-day treatment protocol, the animals are followed in a “post-hoc” continued free choice paradigm. The design of the post-hoc period is influenced by the results of the acute treatment protocol. Paliperidone-treated animals are followed using vehicle alone to assess the rate at which alcohol drinking returned to baseline. Haloperidol treated animals are followed using increasing doses of haloperidol to assess the effect of these higher doses of haloperidol and alcohol drinking.

Anticipated Results. Paliperidone, but not haloperidol or vehicle, decreases alcohol consumption in Syrian golden hamsters. The effect is accompanied by a modest increase in both water and food intake. During the post-hoc period, alcohol drinking gradually returns toward baseline in the paliperidone-treated animals when vehicle is substituted for paliperidone. However, animals treated with increasing doses of haloperidol demonstrate no decrease in drinking during this period.

Anticipated Conclusions. This study demonstrates that the atypical antipsychotic paliperidone but not the typical antipsychotic haloperidol would selectively and reversibly decrease alcohol consumption in the Syrian golden hamster. The effects of paliperidone or other drugs on alcohol drinking can be assessed in the Syrian golden hamster model or with other animal models, particularly other strains of alcohol drinking rodents, such as the alcohol preferring (P) rat.

A number of embodiments of the invention have been described. Nevertheless, it will be understood that various modifications may be made without departing from the spirit and scope of the invention. Accordingly, other embodiments are within the scope of the following claims.

What is claimed is:

1. A method of treating a patient suffering from substance abuse disorder comprising administering to the patient a therapeutically effective dose of a a pharmaceutical selected from the group consisting of paliperidone, its pharmaceutically acceptable acid addition salts, enantiomeric forms or esters thereof effective to reduce alcohol use.

2. The method of any one of claims 1 in which the patient is not being simultaneously being treated for a psychiatric condition other than substance abuse.

3. The method of any one of claims 1-3 in which the medication comprises i idazoxan.

4. The method of any one of claims 2 in which the medication comprises:
(a) a first component selected from the group consisting of paliperidone, its pharmaceutically acceptable acid addition salts, enantiomeric forms and esters thereof and
(b) a second component, which strongly blocks α2 receptor.

5. The method of claim 4 in which the second component is idazoxan.

6. The method of claim 2 in which the medication is formulated as a single dose comprising both the first and the second components.

7. The method of claim 6 in which the medication strongly blocks the α2C receptor.

8. The cocktail of claim 7 in which the second component is idazoxan.
